(19) United States
(12) Patent Application Publication
(10) Pub. No.: US 2026/0137564 A1
(43) Pub. Date: May 21, 2026

(54) REMOTE INCONTINENCE DETECTION AND MONITORING SYSTEM WITH METHOD AND MANUFACTURING METHOD THEREOF

(71) Applicant: Raja Singh Tuli, Montreal (CA)

(72) Inventor: Raja Singh Tuli, Montreal (CA)

(21) Appl. No.: 19/393,829

(22) Filed: Nov. 19, 2025

Related U.S. Application Data
(60) Provisional application No. 63/722,790, filed on Nov. 20, 2024.

(51) Int. Cl. A61F 13/42; A61F 13/15; H01F 27/28

(52) U.S. Cl. CPC A61F 13/42; A61F 13/15699; H01F 27/2804; A61F 2013/424

(57) ABSTRACT

A remote incontinence detection/monitoring system is provided with its methods of operation and manufacturing. The system comprises a diaper embedded with markers that are each associated with a dedicated frequency. Each marker is operable to relay or reflect towards an external receiver, or absorb or interfere with the wireless signal tuned to its dedicated frequency that is emitted from an external transmitter. When in contact with liquid, a marker loses its ability to relay, reflect, absorb, or interfere with the wireless signal tuned to its dedicated frequency, indicating the diaper is saturated on the marker's spot. A method of manufacturing diapers embedding components for said system is provided wherein each diaper manufactured from a same roll of materials will be embedded with the same number of markers reacting to the same serial of pre-set dedicated frequencies with not a single frequency missing or repeating within each and every diaper.

### CROSS-REFERENCE TO RELATED APPLICATIONS

This application claims priority to U.S. provisional patent application No. 63/722,790, entitled “REMOTE INCONTINENCE DETECTION AND MONITORING SYSTEM WITH METHOD AND MANUFACTURING METHOD THEREOF,” filed on Nov. 20, 2024. The content of this U.S. provisional patent application is hereby incorporated by reference in its entirety for all purposes.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM (EFS-WEB)

Not Applicable

### STATEMENT REGARDING PRIOR DISCLOSURES BY THE INVENTOR OR A JOINT INVENTOR

Not Applicable

### BACKGROUND OF THE INVENTION

### Field of the Invention

The present invention relates to a system to remotely detect and monitor incontinence in a diaper-like product with no need for a pod to establish electrical connection between wet sensors.

### Description of the Related Art

With the growing age of the population there is an increased need of adult protection against incontinence like diapers, pads or briefs to name a few. For the purpose of clarity this description will refer to diapers but other incontinence protections are within the scope of this disclosure.

Institutions like hospitals and retirement homes face the challenge of providing care to residents. Such care includes timely changing the resident's diapers, in order to prevent several problems ranging from health issue and patients'comfort, to diapers consumption and laundry management.

One course of action came in the form of diapers including sensors. Those sensors come in different forms of strips. They can be incorporated in the diaper during the diaper manufacturing or by modifying the diaper after. They can also be added to the diaper for instance in the form of sensor strips.

The existing art significantly relies on pods that are clipped or attached to the diaper by various means and which purpose is to relay what the sensors detect to an external unit that will monitor the presence of liquid in the diaper and trigger the alarm for changing it. Some prior art diaper also includes components like electronic chips so the diaper monitors itself and will send a warning to change it.

As for the means to detect and monitor the presence of liquid in the diaper, the prior art mostly relies on the creation of an electric circuit formed by the combination of the sensors, the pod and the liquid released in the diaper. Detection and monitoring range from simply triggering an alarm when the diaper is wet, to triggering the alarm only when the diaper reaches its saturation level.

One shortcoming of electrical circuits formed by the combination of sensors and pods is that variations in the electrical signals be caused by the manufacturing of the sensors or by the way the pods are installed. Indeed, if the pod is not properly installed and connected, the detection and monitoring might not be as efficient as expected. It also requires some training for caregiver to guarantee the sensors and pods are properly installed.

Another prior art wetness detection system is disclosed in US2004/0070510A1, in which a resonant tag is affixed to the diaper whose substantially constant resonant frequency is eliminated when its inductor is contacted by urine and in which a remote detection means emits a substantially constant swept frequency and activates an alarm when the resonant frequency is eliminated. A problem with this type of prior art wetness detection is that it requires a pre-manufactured resonant tag to be affixed to the diaper, which complicates the manufacture of the diaper with such wetness detection system. And with a resonant tag affixed to the diaper, such system can only determine whether the specific location of the resonant tag is contacted by urine, but cannot determine the saturation of the diaper.

Also, another prior art patient incontinence monitoring apparatus and method are disclosed in U.S. Pat. No. 6,774,800, in which a RF tag is positioned in contact with or in spaced relation with the liquid absorbent material and is configured to absorb a wireless excitation signal whereupon a change in the amount of discharged fluid received in the liquid absorbent material causes a change in the absorption of the energy of the wireless excitation signal by the RF tag. This prior art also requires a pre-manufactured resonant tag to be affixed to the diaper, which complicates the manufacture of the diaper with tag.

Thus, it is desirable to provide alternate means to detect and monitor incontinence without resorting to altering the diaper or significantly modifying its manufacturing process. It is desirable to offer a diaper that does not require the manual installation of sensors. It is desirable to offer a diaper that allows the detection and monitoring of incontinence without the need for a pod to relay the data detected by the sensor to the actual monitoring system. It is also desirable to offer a means to detect and monitor incontinence with precision and that is not subject to undesired variations and that does not require to resort to sophisticated components. It is also desirable to provide a cost-effective means to manufacture such a diaper which implies to displace the actual detection and monitoring process out of the diaper.

### SUMMARY OF THE INVENTION

In an aspect, the present disclosure relates to a combination of a diaper with an external monitoring system. During the manufacturing of the diaper, rolls of materials are used and the diapers are cut at a pre-set length and the other features are added to finalize the product. Markers/tags are provided on the material, and along the length of the diaper, in order to maintain a pre-set number of said markers/tags for each diaper. All manufactured diapers have the same number of markers/tags printed inside.

Each marker/tag is configured to react to a determined individual radio frequency and the total number of markers provided in a diaper will always correspond to a pre-determined set of frequencies that will be repeated for every set of markers. Each manufactured diaper contains the same number of markers responding to the same set of pre-determined frequencies. As a result, a series of manufactured diaper will be identical when it comes to the number of markers and the frequencies they correspond to.

An external monitoring system comprises a transmitter and a receiver to send and receive a series of electromagnetic signals corresponding to the pre-set frequencies associated with the markers. As long as a marker is not exposed to a liquid, the signal transmitted from the transmitter at that marker's frequency will be responded by the marker, e.g. be relayed or reflected by the marker towards the receiver or will be absorbed or interfered with by the marker so as not to be received by the receiver. When a marker is finally exposed to the liquid, its ability to relay or reflect or absorb or interfere with the signal is lost and the frequency associated to that particular marker becomes silent or appears at the receiver. As more and more liquid expands into the diaper, and said diaper gets saturated, more markers (if present) lose their ability to react to their respective frequency. Based on the absence or presence of the frequencies at the receiver and their emplacement it becomes possible to determine the moment the diaper has to be changed and alert the caregiver accordingly.

According to an aspect of the present disclosure, a method for providing a resonant component on a layer material is provided that comprises providing at least an inductor portion of the resonant component by using metallic material; and printing other portions of the resonant component.

In an embodiment, the resonant component may be provided on one side of the layer material. As an example, the resonant component may be provided by three stacked layers: a first conductive layer including the inductor portion and one or two plates, a second conductive layer including at least one plates, and an insulation layer interposed therebetween, in which the plate or each of the two plates in the first conductive layer overlaps one of the at least one plate in the second conductive layer to form a capacitor. And, printing other portions of the resonant component may comprise printing a straight conductive line along the layer material's length, as the second conductive layer.

In another embodiment, the resonant component may comprise at least one capacitor whose two plates are respectively provided on two opposite sides of the layer material. As an example, the resonant component may be provided by two conductive layers that are provided respectively on the two opposite sides of the layer material. Among the two conductive layers, the one including the inductor portion may be provided by using metallic material, or the inductor portion may be provided by using metallic material as part of one of the two conductive layers. Among the two conductive layers, the one not including the inductor portion may be printed as a straight conductive line along the layer material's length.

In this aspect, providing the at least an inductor portion of the resonant component by using metallic material may comprise directly stamping the at least an inductor portion of the resonant component with metallic material. Or, providing the at least an inductor portion of the resonant component by using metallic material may comprise transferring the at least an inductor portion of the resonant component from a sheet on which the at least an inductor portion of the resonant component is pre-provided with metallic material. Alternatively, providing the at least an inductor portion of the resonant component by using metallic material may comprise coating with metallic material and then etching the at least an inductor portion.

In another aspect of the present disclosure, a method of manufacturing an absorbent article is provided that comprises: providing multiple layers of the absorbent article to an assembly line from separate rollers supplying separate sheets of materials; providing a resonant component on at least one of the separate sheets; and combining all the multiple layers to form the absorbent article. At least an inductor portion of the resonant component is provided by using metallic material, while other portions of the resonant component is printed.

In an embodiment, the resonant component may be provided on one surface of the corresponding separate sheet. As an example, the resonant component may be provided by three stacked layers: a first conductive layer including the inductor portion and one or two plates, a second conductive layer including at least one plate, and an insulation layer interposed therebetween, in which the plate or each of the two plates in the first conductive layer overlaps one of the at least one plate in the second conductive layer to form a capacitor. And the second conductive layer may be printed as a straight conductive line along the corresponding sheet's length.

In another embodiment, the resonant component may comprise at least one capacitor whose two plates are respectively provided on two opposite surfaces of the corresponding separate sheet. As an example, the resonant component may be provided by two conductive layers that are provided respectively on the two opposite surfaces of the corresponding separate sheet. Among the two conductive layers, the one including the inductor portion may be provided by using metallic material, or the inductor portion may be provided by using metallic material as part of one of the two conductive layers. Among the two conductive layers, the one not including the inductor portion may be printed as a straight conductive line along the corresponding sheet's length.

In this aspect, the at least an inductor portion of the resonant component may be directly stamped. Or, the at least an inductor portion of the resonant component may be provided by transferring the at least an inductor portion of the resonant component from a sheet on which the at least an inductor portion of the resonant component is pre-provided with metallic material. Alternatively, the at least an inductor portion of the resonant component may be provided by coating with metallic material and then etching the at least an inductor portion.

In a further aspect of the present disclosure, an absorbent article is provided that comprises: an absorbent core, and a layer provided with a resonant component. At least an inductor portion of the resonant component is provided by using metallic material while other portions of the resonant component are printed.

In an embodiment, the resonant component may be provided on one surface of the corresponding layer. As an example, the resonant component may be provided by three stacked layers: a first conductive layer including the inductor portion and one or two plates, a second conductive layer including at least one plate, and an insulation layer interposed therebetween, in which the plate or each of the two plates in the first conductive layer overlaps one of the at least one plate in the second conductive layer to form a capacitor. And, the second conductive layer may be a straight conductive line printed along the corresponding layer's length.

In another embodiment, the resonant component may comprise at least one capacitor whose two plates are respectively provided on two opposite surfaces of the corresponding layer. As an example, the resonant component may be provided by two conductive layers that are provided respectively on the two opposite surfaces of the corresponding layer. Among the two conductive layers, the one including the inductor portion may be provided by using metallic material, or the inductor portion may be provided by using metallic material as part of one of the two conductive layers. Among the two conductive layers, the one not including the inductor portion may be a straight conductive line printed along the corresponding sheet's length.

In a still further aspect of the present disclosure, an absorbent article is provided that comprises: an absorbent core, and a layer provided with a metallic component on one side and a printed straight conductive line on the other side. The metallic component comprises an inductor portion and one or two plates, and the printed straight conductive line overlaps the one or two plates to form one or two capacitors.

### BRIEF DESCRIPTION OF THE DRAWINGS

FIG. 1 is a schematic top view of an exemplary diaper according to an embodiment of the present disclosure;

FIG. 2 illustrates a schematic process for printing a marker/tag (e.g. RF tag) with three layers, according to an embodiment of the present disclosure;

FIG. 2A schematically illustrates a material layer/roll on which multiple conductive layers are provided along the length of a material layer/roll and aligned with each other and a single wide conductive line is provided along and across the whole length of the material layer/roll serving as the other conductive layer for each of multiple conductive layers, according to an embodiment of the present disclosure;

FIG. 3 illustrates another schematic process for printing a marker/tag (e.g. RF tag) with three layers, according to an embodiment of the present disclosure;

FIG. 4 illustrates a schematic process for providing a marker/tag (e.g. RF tag) by using metal stamping (and optionally printing), according to an embodiment of the present disclosure;

FIG. 5 illustrates another schematic process for providing a marker/tag (e.g. RF tag) by using metal stamping (and optionally printing), according to an embodiment of the present disclosure.

### DETAILED DESCRIPTION OF THE PREFERRED EMBODIMENTS

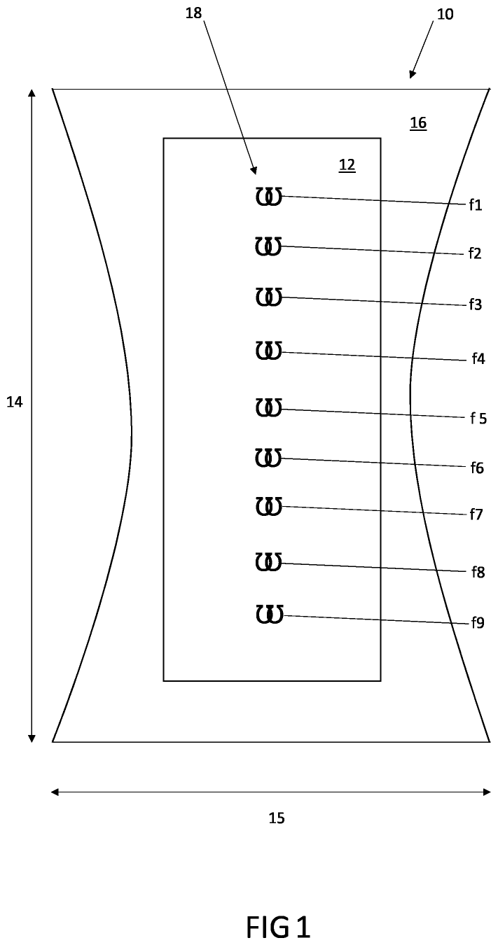

FIG. 1 illustrates an exemplary diaper 10 with a length 14 and a width 15 according to an embodiment of the present disclosure. As appreciated by those skilled in the art, the diaper 10 is primarily composed of at least two layers of material: an absorbent layer 12 whose purpose is to absorb urine or any other body fluids and a liquid-impervious layer 16. It is appreciated that other constructions for diaper are possible and shall be considered to be within the scope of the present disclosure.

In an embodiments of the present disclosure, one or more markers/tags 18 are provided on e.g. the liquid-impervious layer 16. Preferably, the tag(s) 18 is provided on the inner side of the liquid-impervious layer 16 (i.e. the side facing the absorbent layer 12) with the absorbent layer 12 being situated on top of said tags 18, that is, the tag(s) 18 is sandwiched between the inner side of the liquid-impervious layer 16, where they are provided, and the bottom side of the absorbent layer 12. And it is also possible to provide the tag(s) on the outer side of the liquid-impervious layer 16 (i.e. the side opposite to the absorbent layer 12), or on/in another layer(s) of an absorbent article. In the illustrative embodiment as depicted in FIG. 1, nine tags 18 are provided on the central axis of diaper 10 along its length 14 on the inner side of the liquid-impervious layer 16 as an example. It is to be noted that the number (e.g. nine) and the arrangement (e.g. the centered disposition on the inner side of the liquid-impervious layer, the spacing between two adjacent tags) of the tag(s) 18 as illustrated in FIG. 1 are for illustrative purpose only, and in diaper 10 (in particular, on one or more of its layers e.g. on the liquid-impervious layer 16 in the example as illustrated in FIG. 1) there may exist a different number of tags 18 being disposed in other arrangements, e.g. to the left or to the right, on the outer side, more than one column and/or line etc.

As illustrated in FIG. 1, the or each tag 18 is associated with a respective dedicated frequency. In particular, the or each tag 18 is provided or configured such that it operates with a respective dedicated frequency, that is, during normal operation the or each tag is operable to react to (e.g. resonate with and re-transmit, or relay, or reflect, or absorb, or interfere with, etc.) signal tuned to its respective dedicated frequency. As an example, in FIG. 1 from top to bottom, the nine tags 18 are associated with frequencies f1, f2, . . . , f9 respectively. In various embodiments of the present disclosure, the frequencies associated with the tags 18 are different from each other in a diaper, e.g. the frequencies f1, f2, . . . , f9 as illustrated in FIG. 1 are (slightly) different from each other.

Instead of first manufacturing the tags 18 and then inserting them in the diaper or on one or more of its layers e.g. on the liquid-impervious material/layer, the tags 18 may be directly provided or manufactured (e.g. printed, stamped) on a corresponding layer of an absorbent article (e.g. the liquid-impervious material/layer 16) during the absorbent article manufacturing process, i.e. in absorbent article manufacturing assembly line, according to an embodiment of the present disclosure, in order to avoid the significant modification or complication in the absorbent article manufacturing assembly line.

As mentioned above, the tag(s) 18 is provided or configured to operate with their respective dedicated frequencies. In an embodiment of the present disclosure, the or each tag 18 is configured to respond to a signal tuned to its respective dedicated frequency by being energized by that signal and then transmitting a signal at the same frequency back. For example, the tag 18 may be configured to function as a LC resonant circuit with the resonant frequency being its respective dedicated frequency.

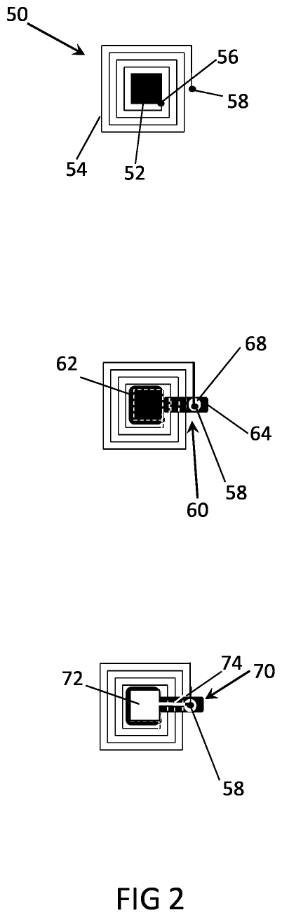

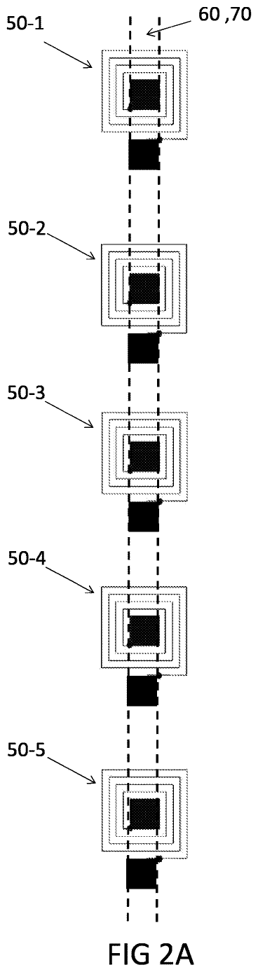

In an embodiment of the present disclosure where the tag 18 functions as a LC resonant circuit, the tag 18 can be provided by providing on a corresponding layer of an absorbent article (e.g. the liquid-impervious material/layer) a capacitor and an inductor, e.g. a center capacitor and an inductor arranged (e.g. as coil) around the capacitor. Please note that the capacitor and the inductor in a LC resonant tag can be arranged in another manner, which also can be used with the technical solutions of the present disclosure. As an example, the tag 18 may be composed of three layers that are printed on e.g. the liquid-impervious material/layer, a first conductive layer 50, a second insulation layer 60, and a third conductive layer 70. FIG. 2 illustrates an exemplary process for providing (e.g. printing) a tag 18 according to an embodiment of the present disclosure.

As illustrated in the upper diagram of FIG. 2, a first conductive layer 50 is printed on e.g. the liquid-impervious material/layer 16, in which a plate 52 is printed at center and a conductor 54 is printed spirally around the center plate 52 with its first terminal 56 being connected to the center plate 52. The conductor 54 also comprises a second terminal 58. It is appreciated that the conductor spirally around the center plate forms a coil, i.e. an inductor.

Next, a second insulation layer 60 is printed over the first conductive layer 50 in order to insulate the plate and the conductor in the first conductive layer 50 from those in the third conductive layer 70 (to be discussed later). In an embodiment of the present disclosure, the second insulation layer 60 covers at least the plate 52 and exposes at least the second terminal 58 and part of the conductor 54, e.g. the second insulation layer 60 only covers the center plate 52, and a connection portion from the center plate 52 to the second terminal 58, with a hole at the location of the second terminal 58 to expose it. For example, in the embodiment as illustrated in the middle diagram of FIG. 2, the second insulation layer 60 is formed of a center insulation portion 62 that covers the center plate 52, and a connection insulation portion 64, with a hole 68 to expose the second terminal 58.

Then a third conductive layer 70 is printed on top of the second insulation layer 60 in which another plate 72 is formed at the location corresponding to the plate 52 (i.e. on the center insulation portion 62), and a conductor 74 is formed on the connection insulation portion 64 to connect the plate 72 with the exposed second terminal 58. It is appreciated that the two plates 52 and 72 separated by the center insulation portion 62 form a capacitor, the conductor 54 spirally around the plate 52 forms an inductor, and the capacitor and the inductor are connected by the conductor 74. It is appreciated that by controlling e.g. the thickness of the center insulation portion 62 (i.e. the distance between the plates 52 and 72) and/or the area of the plates 52 and 72 and/or the number of turns of the coil formed by the conductor 54, a tag 18 can be configured with a desired resonant frequency, i.e. its dedicated frequency. It is also understood that the third conductive layer 70 may be constructed as a single wide conductive line which serves as both the plate 72 (by its portion that overlaps, is opposite to, and corresponds to the plate 52) and the conductor 74 (by its portion that connects the plate 72 to the exposed terminal 58), provided that the second insulation layer 60 is adapted accordingly.

It will be appreciated that the three layers can be printed in an order reversed from that as illustrated in FIG. 2. That is, first in the conductive first layer 50 that is printed on e.g. the liquid impervious layer the plate 72 and the conductor 74 are printed, next the second insulation layer 60 is printed over the first conductive layer 50 with a hole 68 to expose the free terminal (corresponding to the second terminal 58 in FIG. 2) of the conductor 74 (in this case, the second insulation layer 60 will not cover the inductor (i.e. the conductor 52) in the third conductive layer 70, and thus the footprint of the second insulation layer 60 may be larger than that as illustrated in FIG. 2, e.g. the second insulation layer 60 may has a same or even larger footprint as the third conductive layer 70), and then the third conductive layer 70 is printed on top of the second insulation layer in which the plate 52 and the conductor 54 (i.e. inductor) that is connected to the free terminal of the conductor 74 are printed.

In the embodiment as illustrated in FIG. 2, the connection between the second terminal 58 in the conductive first layer 50 and the conductor 74 in the conductive third layer 70 is implemented by a physical (electrical) direct connection, i.e. with a hole 68 in the second insulation layer 60. However, it is understood that there exists other ways to connect a terminal in one layer to a conductor in another layer with an intermediate layer being interpolated between the two layers, e.g. a capacitive connection. Still referring to FIG. 2, a plate may be provided at the second terminal 58 in the conductive first layer 50, and another plate may be provided at the free terminal (corresponding to the second terminal 58 in FIG. 2) of the conductor 74 in the conductive third layer 70 opposite to the plate at the second terminal 58, with a portion of the second insulation layer 60 being sandwiched between these two plates, thereby constituting a capacitor for capacitively connecting the second terminal 58 and the conductor 74 (in particular, its free terminal).

When capacitive connection is used to connect the second terminal 58 and the conductor, the third conductive layer 70 may also be constructed as a wide conductive line which serves as both the conductor 74 and two plates in the third conductive layer 70, 74, similarly as mentioned above. In an embodiment of the present disclosure, the third conductive layer (e.g. 70 in FIG. 2) may be constructed as a wide conductive line extending along and opposite to the line that connects the two plates (e.g. 52 and the one at the terminal 58 in FIG. 2) in the first conductive layer (e.g. 50 in FIG. 2) and overlapping at least partly both plates, which wide conductive line both functions as the conductor (e.g. 74 in FIG. 2) and forms two capacitors respectively with the two plates in the first conductive layer (e.g. one capacitor with the plate 52, and one capacitor with the plate at the terminal 58). As an example, the two plates (e.g. 52 and the one at the terminal 58 in FIG. 2) in the first conductive layer of a resonant circuit (e.g. marker/tag 18 in FIG. 1) may be aligned along the length (e.g. 14 in FIG. 1) of the absorbent article (e.g. 10 in FIG. 1), and a wide conductive line (and also a wide insulation line, if the first and the third conductive layers are provided on the same side of the corresponding layer of the absorbent article) may be provide along and across the absorbent article's whole length, in which case the single wide conductive line across the absorbent article's whole length may serve as third conductive layers (e.g. 70 in FIG. 2) for all resonant circuits (e.g. marker/tag 18 in FIG. 1) aligned and arranged along that line. FIG. 2A schematically illustrates a material layer/roll on which multiple conductive layers (each having at least one plate and a conductor spirally provided around one plate) are provided along the length of a material layer/roll and aligned with each other and a single wide conductive line (and optionally also a single wide insulation line) is provided along and across the whole length of the material layer/roll serving as the other conductive layer (and optionally an insulation layer) for each of multiple conductive layers. As illustrated in FIG. 2A, multiple conductive layers 50-1, 50-2, . . . 50-5 are provided on a material layer/roll, each comprising two plates and a conductor spirally provided around one of the plates. These multiple conductive layers are aligned to each other and arranged along the length of the material layer/roll. And a single wide conductive line 70 and optionally a single wide insulation line 60 are provided that overlap the two plates of every conductive layers 50-1, 50-2, . . . 50-5, thereby forming multiple tags/marks i.e. resonant circuits. It is appreciated that the single wide conductive line along and across the absorbent article's whole length may be replaced with multiple discrete wide lines, each of which is aligned to the respective first conductive layer to form one of the resonant circuits (e.g. marker/tag 18 in FIG. 1). In an embodiment of the present disclosure, the width of the wide conductive line functioning as the third conductive layer may be at least the same as the larger one among the widths of the two plates in the first conductive layer.

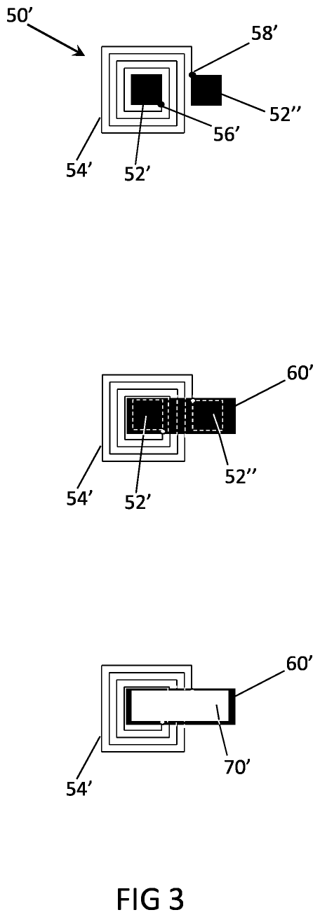

FIG. 3 illustrates an exemplary process for providing (e.g. printing) a tag 18, in which a capacitive connection is employed to connect the first conductive layer and the third conductive layer and a wide conductive line is provided as the third conductive layer, according to an embodiment of the present disclosure. As illustrated in the upper diagram of FIG. 3, in the first conductive layer 50′ all portions (e.g. 52′, 54′, 56′, 58′) are same or similar and thus are indicated with similar reference numbers as that illustrated in FIG. 2, except that an additional plate 52″ may be provided at the second terminal 58′. Next, as the second insulation layer, a wide insulation line 60′ is printed over the first conductive layer 50′, overlapping both plates 52′ and 52″ in the first conductive layer 50′, as illustrated in the middle diagram of FIG. 3. Then, as illustrated in the bottom diagram of FIG. 3, a wide conductive line 70′ is printed on top of the second insulation layer 60′, also overlapping both plates 52′ and 52″ in the first conductive layer 50′, thereby forming two capacitors respectively with the two plates 52′ and 52″ in the first conductive layer 50′ and forming a closed loop of a LC circuit with the first conductive layer 50′. As mentioned above, where multiple LC circuits is to be provided on one layer of an absorbent article, the first conductive layers 50′ of all these LC circuits may be aligned and arrange along the length of the absorbent article, and a single wide insulation line 60′ and a single wide conductive line 70′ may be provided across and along the length of the absorbent article, overlapping both plates 52′ and 52″ of all the first conductive layers 50′, thereby providing all these LC circuits. It is appreciated that, the single wide insulation line 60′ and/or the single wide conductive line 70 may be replaced with multiple discrete wide lines, each of which is aligned to one of the first conductive layers 50′ thereby forming one LC circuits. It is also appreciated that the insulation line 60′ may be omitted if the first conductive layer 50′ and the wide conductive line 70′ are respectively provided on the two opposite side of the corresponding layer of the absorbent article, as detailed below.

Though all the three layers 50/50′, 60/60′, and 70/70′ are described above and illustrated in FIGS. 2 and 3 as being provided (e.g. printed) on a same side of a corresponding layer (e.g. a liquid impervious layer) of an absorbent article (e.g. a diaper), it is also appreciated that the conductive first layer 50/50′ and the conductive third layer 70/70′ may be provided (e.g. printed) on two opposite sides of a layer (e.g. a liquid impervious layer) of an absorbent article (e.g. a diaper), in which case the second insulation layer 60/60′ may be omitted because the corresponding layer serves as an insulation layer between the conductive first layer 50/50′ and the conductive third layer 70/70′. Similarly as the configuration on a same side, the connection between the second terminal 58 in the conductive first layer 50 and the conductor 74 (in particular, its free terminal) in the conductive third layer 70 may be implemented by e.g. a capacitive connection, that is, a plate is provided at the second terminal 58 in the conductive first layer 50, and a corresponding plate is provided at the free terminal of the conductor 74 in the conductive third layer 70 opposite to the plate at the second terminal 58, thereby constituting a capacitor for capacitively connecting the second terminal 58 and the conductor 74. It is further appreciated that a via hole may be provided in the corresponding layer through which the second terminal 58 on one side of the layer is physically (electrically) connected to the conductor 74 (in particular, its free terminal) in the opposite side of the same layer, similarly as those as illustrated in FIG. 2.

In the embodiment in which a tag is printed e.g. as illustrated in FIGS. 2 and 3, the conductive layers (e.g. the conductive first layer 50/50′ and the third conductive layer 70/70′ as illustrated in FIGS. 2 and 3) are printed with conductive material such as carbon ink or silver. It is understood that carbon ink has a high resistivity/resistance, while silver is expensive and has a resistivity/resistance that is lower than carbon ink but still much higher than metal. It is further understood that a resonant circuit such as a RF tag or LC resonant circuit e.g. marker/tag as illustrated in FIGS. 1-3 cannot work very well with a high resistance, because in response to a signal with its resonant frequency, the resonant circuit with a high resistance resonates and e.g. transmits back a response signal with the same resonant frequency, which response signal has however a small amplitude due to the high resistance of the resonant circuit and thus fades very soon and in turn cannot be detected very well or even cannot be detected at all. Therefore it is desirable for the resonant circuit e.g. mark(s)/tag(s) as illustrated in FIGS. 1-3 to have a low resistance or a high conductivity.

It is appreciated that the conductor that forms inductor (e.g. conductor 54/54′ as illustrated in FIGS. 2 and 3) is a thin and long conductive line, and thus its resistivity/resistance has a great impact on the performance of the resonant circuit (e.g. marker/tag as illustrated in FIGS. 2 and 3). Accordingly, in an embodiment of the present disclosure, at least an inductor portion of a resonant circuit may be manufactured with metallic material such as metal e.g. copper or aluminum, instead of being printed with carbon ink or silver. As an example, in a resonant circuit (e.g. marker/tag 18 as illustrated in FIG. 1) an inductor (formed by a spiral conductor, e.g. 54/54′ in FIGS. 2 and 3) or a whole conductive layer (e.g. 50/50′ as illustrated in FIGS. 2 and 3) including an inductor (e.g. including a plate 52/52′ and an inductor 54/54′ in FIGS. 2 and 3) may be produced with metallic material e.g. on a layer of an absorbent article, for example by means of a metal stamping process, in an embodiment of the present disclosure. With reference back to FIGS. 2 and 3, inductor 54/54′, or the whole first conductive layer 50/50′ may be produced with metallic material e.g. on a layer of an absorbent article, for example by means of a metal stamping process.

As for the other conductor (e.g. conductor 74 as illustrated in FIG. 2) that does not form inductor but only connects capacitor (in particular, one of its plate) to inductor, its resistivity/resistance does not have much impact on the performance of the resonant circuit (e.g. marker/tag as illustrated in FIG. 2) because it is of a short distance and can be made thick/wide. Accordingly, such conductor (e.g. conductor 74 as illustrated in FIG. 2) that does not form inductor may be printed e.g. with carbon ink or silver, just like in the process as illustrated in FIG. 2. Similarly, the resistivity/resistance of the wide conductive line (e.g. 70′ in FIG. 3) functioning as the third conductive layer does not have much impact on the performance of the resonant circuit because of its wide configuration and short distance, and thus also may be printed e.g. with carbon ink or silver. Also, the other portions (e.g. the second insulation layer 60, the third conductive layer 70 such as the plate(s) in the third layer, and even the plate 52 in the first conductive layer 50 (if not produced with metallic material)) of the resonant circuit (e.g. marker/tag 18) may be printed e.g. with carbon ink or silver or insulation material, just like in the process as illustrated in FIG. 2.

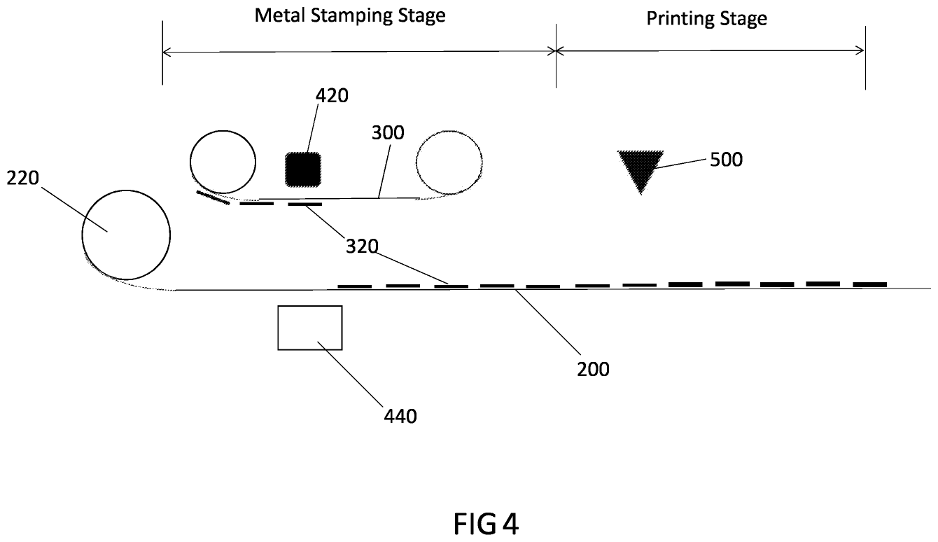

An assembly line used for manufacturing an absorbent article e.g. a diaper is well known in the prior art. Generally such assembly processes are known as roll-to-roll process as multiple rolls of materials are combined one on top of other and combined to form a final product. FIG. 4 is a schematic view of a metal stamping stage and a printing stage in an absorbent article (e.g. diaper) assembly line, used for providing a marker/tag (e.g. resonant circuit) onto a layer material (e.g. the liquid-impervious layer material) of an absorbent article in an absorbent article assembly process, in which an inductor or a whole conductive layer (including an inductor and a plate of a capacitor that are connected to each other) is metal stamped onto the layer material, according to an embodiment of the present disclosure. As illustrated in FIG. 4, a layer material 200 (e.g. the liquid-impervious layer material) of an absorbent article is supplied in a form of a web of long sheet roll in the assembly line using a roller equipment 220. Another roll of sheet 300 (e.g. a plastic sheet) with metallic components 320 (that is, inductors e.g. inductor 54/54′ as illustrated in FIGS. 2 and 3 or conductive layers e.g. 50/50′ in FIGS. 2 and 3) being pre-manufactured thereon with metallic material is provided in parallel to the layer material 200, with the inductors or conductive layers 320 on the sheet 300 facing towards one side of the layer material 200. And a stamper head 420 and a support surface 440 are provided to the other side of the sheet 300 and the layer material 200 respectively (i.e. the stamper head 420 and the layer material 200 are respectively provided to two opposite sides of the sheet 300, while the sheet 300 and the support surface 440 are respectively provide to two opposite sides of the layer material 200), and opposite to each other (i.e. the stamper head 400 is opposite to the support surface 400). In this way, the metallic components 320 (that is, the inductors e.g. inductor 54/54′ in FIGS. 2 and 3 or conductive layers e.g. the first conductive layer 50/50′ in FIGS. 2 and 3) pre-manufactured with metallic material on the sheet 300 may be metal stamped (i.e. transferred) by the stamper head 420 e.g. by means of heat, pressure, vibration, ultrasound, etc. onto the layer material 200 during the manufacturing process of an absorbent article e.g. a diaper.

It is appreciated that the metallic components 320 (i.e. inductor e.g. inductor 54/54′, or conductive layer with an inductor and a plate of a capacitor being connected to each other e.g. first conductive layer 50/50′) may be pre-manufactured on the sheet 300 with metallic material such as metal e.g. copper or aluminum in various ways. As an example, a coating of metallic material may be provided on a sheet 300 and then be patterned with mechanic means (e.g. cut, etched, stamped etc.) to form the metallic components (e.g. inductors or conductive layers), thereby obtaining the sheet 300 as illustrated in FIG. 3.

After metallic inductor(s) or conductive layer(s) has been stamped onto the layer sheet 200, the layer sheet 200 with metallic inductor(s) or conductive layer(s) 320 proceeds to a printing stage, in which other portions of the resonant circuit (e.g. marker/tag 18) are printed onto the layer material 200 by at least one printing head 500 (for conductive portions, with e.g. carbon ink or silver, while for insulation portion, with insulation material), thereby providing resonant circuit(s) (e.g. marker(s)/tag(s) 18) on the layer material 200, which then is supplied to the assembly line of an absorbent article e.g. a diaper to manufacture an absorbent article.

In the printing stage, the other portions of the resonant circuit (e.g. marker/tag 18) may be printed in the same way as that in FIG. 2, with the exception that its inductor (e.g. 54/54′ in FIGS. 2 and 3) or its first conductive layer (e.g. 50/50′ in FIGS. 2 and 3) has already been stamped with metallic material.

Similarly as in FIGS. 2 and 3, the other portions of the resonant circuit may be printed on the same side of the layer material 200 as its inductor or its first conductive layer that is stamped. That is, where the whole first conductive layer 50/50′ is already stamped with metallic material on the layer material 200, in the printing stage the single wide insulation line 60′ or the second insulation layer 60 that covers at least the plate 52 and exposes at least the second terminal 58 and part of the conductor 54 is printed over the first conductive layer 50/50′, and then the single wide conductive line 70′ or the third conductive layer 70 is printed on top of the second insulation layer 60/60′ (in the third conductive layer 70, another plate 72 is formed at the location corresponding to the plate 52 and a conductor 74 is formed on the connection insulation portion 64 to connect the plate 72 with the exposed second terminal 58). On the other hand, if only inductor portion is stamped with metallic material on the layer material 200, the plate(s) (e.g. 52, 52′, 52″ connected to one of its terminal (e.g. 56, 56′, 58′ in FIGS. 2 and 3)) needs to be first printed on the layer material 200, then the second insulation layer 60/the single wide insulation line 60′ and the third conductive layer 70/the single wide conductive line 70′ are printed sequentially. In addition, as mentioned above, the manufacturing order of the resonant circuit (in particular, its three layers) may be reversed, in which case all portions other than the inductor portion or a whole conductive layer (e.g. 50/50′ in FIGS. 2 and 3) including the inductor portion are printed first, and then the inductor portion or the whole conductive layer including the inductor portion is then stamped with metallic material.

It is also appreciated that its two conductive layers (e.g. 50/50′ and 70/70′ in FIGS. 2 and 3) may be provided on two opposite sides of the layer material 200 respectively, i.e. the first conductive layer including metallic inductor as stamped (e.g. 50/50′) and the third conductive layer (e.g. 70/70′) are provided on the two opposite sides of the layer material 200 respectively, and thus its second insulation layer e.g. 60/60′ may be omitted, similar as the process as described above with respect to FIGS. 2 and 3. That is, where the whole first conductive layer 50/50′ is stamped with metallic material on one side of the layer material 200, in the printing stage the third conductive layer 70/70′ is printed on the other side of the layer material 200. On the other hand, if only inductor portion is stamped with metallic material on the layer material 200, the plate(s) (e.g. 52, or 52′ and 58′) connected to one of its terminals (e.g. 56 in FIG. 2) needs to be printed on the same side of the layer material 200, and the third conductive layer 70/70′ is printed on the other side of the layer material 200. Similarly as mentioned above, the manufacturing order of the resonant circuit (in particular, its two conductive layers in this case) may be reversed, in which case all portions other than the inductor portion or the whole conductive layer (e.g. 50/50′ in FIGS. 2 and 3) including the inductor portion are printed first, and then the inductor portion or the whole conductive layer including the inductor portion is then stamped with metallic material.

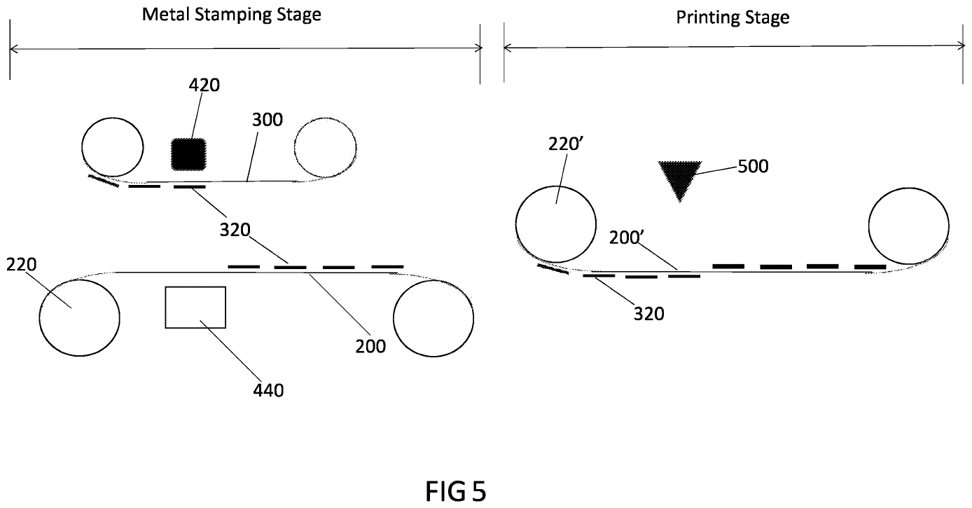

It is appreciated that the metal stamping stage and the printing stage may be provided separately, in an absorbent article assembly line. FIG. 5 is a schematic view of a metal stamping stage and a printing stage in an absorbent article (e.g. diaper) assembly line, used for providing a marker/tag (e.g. resonant circuit) onto a layer material (e.g. the liquid-impervious layer material) of an absorbent article in an absorbent article assembly process, according to an embodiment of the present disclosure. As illustrated in FIG. 5, metallic components 320 are metal stamped onto the layer material 200, similarly as in FIG. 4, that does however not directly proceed to the printing stage as illustrated in FIG. 4. Instead, the layer material 200 with metallic components 320 is rolled as a roll 200′, that is brought into a separate printing stage that is similar to that as illustrated in FIG. 4, to producing the resonant components on the layer material. As can be seen from FIG. 5, the printing may be conducted on the other side of the layer material than the metallic components, and the layer material after the printing stage may further be rolled as another roll and then be brought to the next stage of the assembly line. However, it is also appreciated that the layer material after the printing stage may proceed directly to the next stage of the assembly line.

It is to be noted that the metallic component(s) i.e. at least the inductor portion or the whole conductive layer including an inductor of a resonant circuit may be directly stamped, e.g. onto the corresponding material layer/roll of an absorbent article or onto the corresponding insulation layer of the corresponding resonant circuit, preferably continuously. Alternatively, the metallic component(s) may be provided by coating the corresponding material layer/roll of an absorbent article or the corresponding insulation layer of the corresponding resonant circuit with metal e.g. copper or aluminum and then etching e.g. with standard etching techniques.

What is claimed is:

1. A method for providing a resonant component on a layer material, comprising:
providing at least an inductor portion of the resonant component by using metallic material; and
printing other portions of the resonant component.

2. The method according to claim 1, wherein the resonant component is provided on one side of the layer material.

3. The method according to claim 2, wherein the resonant component is provided by three stacked layers, a first conductive layer including the inductor portion and one or two plates, a second conductive layer including at least one plates, and an insulation layer interposed therebetween, and wherein the plate or each of the two plates in the first conductive layer overlaps one of the at least one plate in the second conductive layer to form a capacitor.

4. The method according to claim 3, wherein printing other portions of the resonant component comprises printing a straight conductive line along the layer material's length, as the second conductive layer.

5. The method according to claim 1, wherein the resonant component comprises at least one capacitor whose two plates are respectively provided on two opposite sides of the layer material.

6. The method according to claim 5, wherein the resonant component is provided by two conductive layers that are provided respectively on the two opposite sides of the layer material.

7. The method according to claim 6, wherein among the two conductive layers, the one including the inductor portion is provided by using metallic material, or wherein the inductor portion is provided by using metallic material as part of one of the two conductive layers.

8. The method according to claim 6, wherein among the two conductive layers, the one not including the inductor portion is printed as a straight conductive line along the layer material's length.

9. The method according to claim 1, wherein providing the at least an inductor portion of the resonant component by using metallic material comprises directly stamping the at least an inductor portion of the resonant component with metallic material.

10. The method according to claim 1, wherein providing the at least an inductor portion of the resonant component by using metallic material comprises transferring the at least an inductor portion of the resonant component from a sheet on which the at least an inductor portion of the resonant component is pre-provided with metallic material.

11. The method according to claim 1, wherein providing the at least an inductor portion of the resonant component by using metallic material comprises coating with metallic material and then etching the at least an inductor portion.

12. A method of manufacturing an absorbent article, comprising:
providing multiple layers of the absorbent article to an assembly line from separate rollers supplying separate sheets of materials;
providing a resonant component on at least one of the separate sheets, wherein at least an inductor portion of the resonant component is provided by using metallic material, while other portions of the resonant component are printed;
combining all the multiple layers to form the absorbent article.

13. The method according to claim 12, wherein the resonant component is provided on one surface of the corresponding separate sheet.

14. The method according to claim 13, wherein the resonant component is provided by three stacked layers, a first conductive layer including the inductor portion and one or two plates, a second conductive layer including at least one plate, and an insulation layer interposed therebetween, and wherein the plate or each of the two plates in the first conductive layer overlaps one of the at least one plate in the second conductive layer to form a capacitor.

15. The method according to claim 14, wherein the second conductive layer is printed as a straight conductive line along the corresponding sheet's length.

16. The method according to claim 12, wherein the resonant component comprises at least one capacitor whose two plates are respectively provided on two opposite surfaces of the corresponding separate sheet.

17. The method according to claim 16, wherein the resonant component is provided by two conductive layers that are provided respectively on the two opposite surfaces of the corresponding separate sheet.

18. The method according to claim 17, wherein among the two conductive layers, the one including the inductor portion is provided by using metallic material, or wherein the inductor portion is provided by using metallic material as part of one of the two conductive layers.

19. The method according to claim 17, wherein among the two conductive layers, the one not including the inductor portion is printed as a straight conductive line along the corresponding sheet's length.

20. The method according to claim 12, wherein the at least an inductor portion of the resonant component is directly stamped.

21. The method according to claim 12, wherein the at least an inductor portion of the resonant component is provided by transferring the at least an inductor portion of the resonant component from a sheet on which the at least an inductor portion of the resonant component is pre-provided with metallic material.

22. The method according to claim 12, wherein the at least an inductor portion of the resonant component is provided by coating with metallic material and then etching the at least an inductor portion.

23. An absorbent article, comprising:
an absorbent core, and
a layer provided with a resonant component, in which at least an inductor portion of the resonant component is provided by using metallic material while other portions of the resonant component are printed.

24. The absorbent article according to claim 23, wherein the resonant component is provided on one surface of the corresponding layer.

25. The absorbent article according to claim 24, wherein the resonant component is provided by three stacked layers, a first conductive layer including the inductor portion and one or two plates, a second conductive layer including at least one plate, and an insulation layer interposed therebetween, and wherein the plate or each of the two plates in the first conductive layer overlaps one of the at least one plate in the second conductive layer to form a capacitor.

26. The absorbent article according to claim 25, wherein the second conductive layer is a straight conductive line printed along the corresponding layer's length.

27. The absorbent article according to claim 23, wherein the resonant component comprises at least one capacitor whose two plates are respectively provided on two opposite surfaces of the corresponding layer.

28. The absorbent article according to claim 27, wherein the resonant component is provided by two conductive layers that are provided respectively on the two opposite surfaces of the corresponding layer.

29. The absorbent article according to claim 28, wherein among the two conductive layers, the one including the inductor portion is provided by using metallic material, or wherein the inductor portion is provided by using metallic material as part of one of the two conductive layers.

30. The absorbent article according to claim 28, wherein among the two conductive layers, the one not including the inductor portion is a straight conductive line printed along the corresponding sheet's length.

31. An absorbent article, comprising:
an absorbent core, and
a layer provided with a metallic component on one side and a printed straight conductive line on the other side, wherein the metallic component comprises an inductor portion and one or two plates, and wherein the printed straight conductive line overlaps the one or two plates to form one or two capacitors.
